(19) United States
(12) Patent Application Publication
(10) Pub. No.: US 2023/0338396 A1
(43) Pub. Date: Oct. 26, 2023

(54) CANNABIDIOL AS A THERAPEUTIC MODALITY FOR COVID-19

(71) Applicant: AUGUSTA UNIVERSITY RESEARCH INSTITUTE, INC., Augusta, GA (US)

(72) Inventors: Babak BABAN, Augusta, GA (US); Krishnan M. DHANDAPANI, Augusta, GA (US); Jack YU, Augusta, GA (US)

(73) Assignee: AUGUSTA UNIVERSITY RESEARCH INSTITUTE, INC., Augusta, GA (US)

(21) Appl. No.: 17/998,790

(22) PCT Filed: May 14, 2021
(86) PCT No.: PCT/US2021/032365

§ 371 Date: Nov. 14, 2022

Related U.S. Application Data
(60) Provisional application No. 63/024,706, filed on May 14, 2020.

(51) Int. Cl. A61K 31/00; A61P 11/00; A61P 31/14

(52) U.S. Cl. CPC A61K 31/658; A61P 11/00; A61P 31/14

(57) ABSTRACT

Compositions and methods for treating or reducing symptoms of COVID-19 are provided herein. An exemplary method includes administering to the subject an effective amount of cannabidiol to reduce acute respiratory distress syndrome caused by COVID-19.

### CROSS-REFERENCE TO RELATED APPLICATIONS

This application claims benefit of and priority to U.S. Provisional Pat. Application 63/024,706 filed on May 14, 2020, and where permitted all of which are incorporated by reference in their entireties.

### STATEMENT REGARDING FEDERALLY SPONSORED RESEARCH

This invention was made with government support under NS110378 awarded by the National Institutes of Health. The government has certain rights in the invention.

### TECHNICAL FIELD OF THE INVENTION

This invention is generally related to compositions and methods of treating coronavirus.

### BACKGROUND OF THE INVENTION

COVID-19 pandemic has profoundly affected human life, inducing high patient morbidity and mortality while stressing health care systems worldwide. SARS-CoV-2, the highly infectious agent responsible for the COVID-19 pandemic, is a novel coronavirus that utilizes a gly-cosylated spike protein to enter human cells via the angiotensin-con-verting enzyme 2 (ACE2) receptor. The lung is a primary site of entry for SARS-CoV-2, as evidenced by massive pulmonary inflammation and development of acute respiratory distress syndrome (ARDS) (Saxena, SK., et al., Coronavirus Dis.;2019:1 (2020)). ARDS is a serious inflammatory lung condition responsible for the highest rate of medical complications and mortality among critically ill patients (Gan, T., et al., Front Microbiol, 21;9:3174 (2018)). In the case of viral respiratory infections, symptoms are usually mild, self-limiting, and confined to the upper airways. However, in more severe respiratory cases, as seen during the COVID-19 pandemic, the infection can affect the lower airways, causing ARDS, increased pulmonary vascular permeability, hypoxemia, increased coagulation, fibrinolysis factors, endothelial and epithelial damages (Ferguson, ND., et al., Intensive Care Med, 38(10):1573 (2019); Spadaro, S., et al., J Inflamm (London), 15;16:1 (2019)). In patients with severe COVID-19, the transition to ARDS condition is mainly due to the occurrence of a cytokine storm and hyperinflammatory responses, including massive production of pro-inflammatory cytokines such as IL-6 and IL-1β, as well as infiltration of neutrophils and monocytes into the lung tissue (Spadaro, S., et al., J Inflamm (London), 15;16:1 (2019); Ghadimi-Moghadam, A., et al., J Biomed Phys Eng, 10(2):241 (2020); Ye., Q., et al., J Infect, pii: S0163-4453(20)30165 (2020)). The cytokine storm contributes to diffuse alveolar damage, alveolar capillary leakage, severe hypoxaemia, intense pulmonary oedema and pulmonary fibrosis (Villar, J., et al., Chest, 155:587 )2019). Currently, other than supportive measures there is no definitive cure for ARDS (Shen, C., et al., JAMA, Mar 27 (2020); Chaari, L., et al., EPMA J, 25:1-6 (2020)), illustrating the urgent need for creative and effective therapeutic modalities to treat this complex condition.

Numerous studies report that cannabinoids may function as immune modulators, limiting the adverse effects of inflammatory diseases (Pini, A., et al., Curr Drug Targets, 13(7):984 (2012)). Endocannabinoids are produced in the respiratory system and cannabinoids-induced bronchodialation suggest a significant therapeutic potential for cannabinoids in the treatment of respiratory diseases, including ARDS in case of patients with severe form of COVID-19 (Bozkurt, TE., Molecules, 24(24). pii: E4626 (2019)). Importantly, several reports demonstrated that cannabidiol (CBD), a phytocannabinoid produced by Cannabis plant, can block IL-6 in several models of inflammatory diseases (Bozkurt, TE., Molecules, 24(24). pii: E4626 (2019)). Further, it is documented that IL-6 production was significantly reduced in the LPS-stimulated peritoneal macrophages, in pancreas during acute pancreatitis as well as in bronchoalveolar lavage fluid in LPS-induced pulmonary inflammation (Nichols, J.M, et al., Cannabis Cannabinoid Res, 5(1):12 (2020)). Therefore, it is very plausible to investigate whether cannabinoids can be considered as therapeutic agents to treat severe viral respiratory infections including COVID-19 and ARDS symptoms.

It is an object of the invention to provide compositions and methods of treating or reducing Acute respiratory distress syndrome and other inflammatory conditions associated with COVID-19.

### SUMMARY OF THE INVENTION

Disclosed herein are cannabinoid based compositions and methods of their use to treat or reduce symptoms associated with viral infections including but not limited to coronaviruses such as SARS-CoV-2 infection or COVID-19. Exemplary cannabinoid based compositions that can be used in the disclosed methods include, but are not limited to, tetrahydrocannabinols (THC), preferably delta-9-tetrahydrocannabinol and delta-8-tetrahydrocannabinol, cannabidiol (CBD), cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV) and cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof. One embodiment provides administering an effective amount of a cannabidiol based composition, for example CBD, to a subject in need thereof to treat or reduce COVID-19 and/or ARDS symptoms. In one embodiment, the disclosed cannabidiol compositions ameliorate the conditions associated with ARDS by reducing inflammation in the lung or airways, reducing inflammatory indices, limiting damage in the lung, and improving the functional capacity of airways.

Another embodiment provides a method of reducing inflammatory symptoms of COVID-19 by administering to a subject in need thereof an amount of cannabidiol effective to reduce inflammation in the subject including, but not limited to inflammation in the lung or airways.

Another embodiment provides a method of reducing ARDS in a subject in need thereof by administering to the subject an amount of cannabidiol effective to reduce inflammation in the subject. In some embodiments cannabidiol reduces the level of inflammatory cytokines including, but not limited to interleukin (IL)-2, IL-7, IL-6, IL-10, tumor necrosis factor (TNF), IFNγ, granulocyte colony-stimulating factor (G-CSF), monocyte chemoattractant protein-1 (MCP1; also known as CCL2), macrophage inflammatory protein 1 alpha (MIP1α; also known as CCL3), CXC-chemokine ligand 10 (CXCL10), C-reactive protein, ferritin, and D-dimers in blood upon SARS-CoV-2 infection. Of note, among the elevated inflammatory mediators, the blood IL-6 level is highly correlated with the disease mortality when COVID-19 survivors and non-survivors are compared, suggesting that fatal COVID-19 is characterized as a cytokine release syndrome (CRS) that is induced by a cytokine storm with high mortality (Shintaro Hojyo, et al., Inflamm Regen. 2020; 40: 37). The inflammatory cytokines can be circulating cytokines or pulmonary cytokines. In some embodiments cannabidiol treatment reduces inflammatory damage to the lungs and improves the functional capacity of the lungs. In one embodiment, the acute respiratory distress syndrome is caused by COVID-19.

Another embodiment provides a pharmaceutical composition containing an effective amount of a cannabinoid to reduce inflammation in a subject in need thereof. The pharmaceutical composition can be formulated for pulmonary administration, nasal administration, or aerosol administration. In one embodiment the cannabinoid is cannabidiol. In other embodiments, the pharmaceutical composition contains one or more cannabinoid selected from the group consisting of tetrahydrocannabinols (THC), preferably delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV), cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

Still another embodiment provides a method of reducing pulmonary inflammation in a subject in need thereof by administering to the subject an effective amount of a cannabinoid to reduce the pulmonary inflammation. In one embodiment the cannabinoid is cannabidiol. In other embodiments, the the cannabinoid is selected from the group consisting of tetrahydrocannabinols (THC), preferably delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV) and cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof. In some embodiments, the subject is infected with a coronavirus including but not limited to SARS-CoV-2. In some embodiments, the subject has ARDS.

Yet another embodiment provides a method for treating or reducing a cytokine storm in a subject in need thereof comprising administering an effective amount of a cannabinoid to treat or reduce the cytokine storm. In one embodiment the cannabinoid is cannabidiol. In other embodiments, the cannabinoid is selected from the group consisting of tetrahydrocannabinols (THC), preferably delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV) and cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

### BRIEF DESCRIPTION OF THE DRAWINGS

FIGS. 1A-1M show that CBD improves lung structure and function following intranasal Poly(I:C) treatment. FIG. 1A is a bar graph showing the effect of intranasal administration of Poly(I:C) and Poly(I:C) + CBD treatment on blood oxygen saturation. FIGS. 1B-1G show histological analysis (H&E) of normal lung tissue (FIGS. 1B-1C) as compared to Poly(I:C) (FIGS. 1D-1E), and Poly(I:C) + CBD treated mice (FIGS. 1F-1G). FIGS. 1H, 1J, and 1L show immunohistochemical analysis of the expression level of IL-6 in in Poly(I:C) (FIG. 1J) and Poly(I:C) + CBD (FIG. 1L) treated lung and compared to normal tissue (FIG. 1H). FIGS. 1I, 1K, and 1M show immunohistochemical staining for neutrophils (Gr1+LY6G+) lung tissue treated with Poly(I:C) (FIG. 1K) and Poly(I:C) + CBD (FIG. 1M) as compared to the untreated normal lung tissue (FIG. 1I).

FIGS. 2A-2Q show anti-inflammatory effect of CBD after intranasal Poly(I:C) treatment. FIGS. 2A-2G show flow cytometry analysis panels demonstrating the effect of on lymphopenia (FIGS. 2A-2C) and IL-6 production (FIGS. 2D-2F) production when mice are treated through intranasal administration of Poly(I:C) (FIGS. 2B, 2E), and Poly(I:C) + CBD (FIGS. 2C, 2F) on as compared to control shams (FIGS. 2A, 2D). FIG. 2G is a bar graph showing the effect of Poly(I:C) and Poly(I:C) + CBD treatment on T cells (**p<0.01) and IL-6 levels (*p<0.05) in the blood compared to sham controls. FIGS. 2H-2Q are flow cytometry analysis panels showing the effects of Poly(I: C) and Poly(I:C) + CBD treatment on the level of inflammatory cytokines (e.g., IL-6, TNFα, IFNγ) (*p<0.05).

FIGS. 3A-3H show CBD improved the symptoms of Poly(I:C)-induced ARDS and normalized the expression level of apelin in the blood. FIGS. 3A-3F are dot plot flow cytometry panels showing the effect of intranasal administration of Poly(I:C) (FIGS. 3C-3D) and Poly(I:C) + CBD (FIGS. 3E-3F) treatment on T-cells and neutrophils in blood samples as compared to the sham control group (FIGS. 3A-3B). FIGS. 3G-3H are a histogram graph (FIG. 3G) and a bar graph (FIG. 3H) showing apelin expression in whole blood of mice as assessed by flow cytometry. The bar graphs are representing the average of values for 10 mice per group (**P <0.03)

FIGS. 4A-4R show CBD improved the symptoms of Poly(I:C)-induced ARDS and normalized the apelin expression in the lung tissues. FIGS. 4A-4F are histological panels showing Masson’s trichrome analysis of normal lung tissue (FIGS. 4A-4B), a high dose of Poly(I:C) (FIGS. 4C-4D), and Poly(I:C) + CBD (FIGS. 4E-4F). FIGS. 4G-4R show immunofluorescence analysis of Apelin expression in normal lung tissue (FIGS. 4G-4J), as compared to lung tissue treated with Poly(I:C) (FIGS. 4K-4N) and Poly(I:C) + CBD (FIGS. 4O-4R).

FIGS. 5A-5M show inhaled CBD was able to prevent further destruction of lung during Acute Lung Injury (ALI) in a murine model. FIGS. 5A-5L are H&E panels of normal lung tissue (FIGS. 5A-5D) as compared to Poly(I:C) ARDS simulated lung tissue (FIGS. 5E-5H), and Poly(I:C) + CBD treated lung tissue (FIGS. 5I-5L). FIGS. 5C, 5G, and 5K show immunohistochemical analysis of the expression level of apelin in Poly(I:C) (FIG. 5G) and Poly(I:C) + CBD (FIG. 5K) treated lung and compared to normal tissue (FIG. 5C). FIGS. 5D, 5H, and 5L show immunohistochemical analysis of the expression level of IL-6 in in Poly(I:C) (FIG. 5H) and Poly(I:C) + CBD (FIG. 5L) treated lung and compared to normal tissue (FIG. 5D). FIG. 5M is a bar graph showing the effect of Poly(I:C) and Poly(I:C) + inhaled CBD treatment on blood oxygen saturation.

FIGS. 6A-6I show inhaled CBD was able to reinstate the frequencies of T cells, reversing the ARDS/ALI-induced lymphoenia both systemically (in the preipheral blood) as well as locally in the lung tissue. FIG. 6 are flow cytometry analysis panels showing the effects of Poly(I:C) and Poly(I:C) + inhaled CBD treatment on the level of on lymphopenia and neutrophils (FIGS. 6A, 6D, 6G) and IL-6 production (FIGS. 6B, 6E, 6H) and IL-10 (FIGS. 6C, 6F, 6I) production when mice are treated with Poly(I:C) (FIGS. 6D-6F), and Poly(I:C) + inhaled CBD (FIGS. 6G-6I) on as compared to control shams (FIGS. 6A-6C).

FIGS. 7A-7F show that inhaled CBD was able to further down regulate IL-6 production and decreased activated infiltrating leukocytes compared in CBD injection. FIGS. 7A-7C are dot plot flow cytometry panels showing the effect of treatment with Poly(I:C) (FIG. 7A), Poly(I:C) + injected CBD (FIG. 7B) and Poly(I:C) + inhaled CBD (FIG. 7C) treatment on activated infiltrating leukocytes. FIGS. 7D-7F are graphs showing the effect of treatment with Poly(I:C) (FIG. 7D), Poly(I:C) + injected CBD (FIG. 7E) and Poly(I:C) + inhaled CBD (FIG. 7F) treatment on IL-6 production in lung tissue.

FIGS. 8A-8C are panels showing that the use of CBD inhalers by healthy young and older subjects had no negative effects on either the T cells frequencies or functionality. Health subjects were treated with 20 mg of CBD for 120 min (FIGS. 8B, 8C) compared to untreated subjects (FIGS. 8A, 8C).

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/- 10%; in other embodiments the values may range in value either above or below the stated value in a range of approx. +/- 5%; in other embodiments the values may range in value either above or below the stated value in a range of approx. +/-2%; in other embodiments the values may range in value either above or below the stated value in a range of approx. +/- 1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “cannabidiol (CBD)” refers to the major nonpsychotropic cannabinoid compound derived from the plant Cannabis sativa, commonly known as marijuana. CBD has been reported to have antioxidant, anti-inflammatory and neuroprotective effects, which occur independent of the canonical cannabinoid CB1 and CB2 receptors.

Coronaviruses are a group of related RNA viruses that cause diseases in mammals and birds. In humans, these viruses cause respiratory tract infections that can range from mild to lethal. Mild illnesses include some cases of the common cold, while more lethal varieties can cause SARS, MERS, and COVID-19.

### II. Cannabidiol as a Therapeutic Modality for COVID-19

In the absence of effective antivirals, the pandemic of COVID-19 remains the most significant challenge to the healthcare system in decades. There is an urgent need for definitive therapeutic intervention. Clinical reports indicate the cytokine storm associated with acute respiratory distress syndrome (ARDS) is the leading cause of mortality in severe cases of COVID-19. Disclosed herein are cannabidiol (CBD) compositions and methods of their use as a therapeutic modality for COVID-19.

### A. Cannabinoid Compositions

Cannabidiol (CBD) is a non-psychotropic phytocannabinoid that regulates immune responses in multiple experimental disease models, including studies showing a benefit following ARDS-like injury in mice (Khodadadi, H., et al., J. Cannabis Cannabinoid Res., 5(3):10 (2020)). Consistent with these findings, a recent commentary, based on anecdotal reports, supports the therapeutic use of CBD in COVID-19-infected patients (Esposito, G., et al., Br J Pharmacol., 10(10):15157 (2020)). While a number of mechanisms are postulated to mediate the anti-viral benefits of CBD, including down-regulation SARS-CoV-2 receptors in human epithelia and suppression of pro-inflammatory cytokine production (e.g., interleukin-1β (IL-1β), IL-6, tumour necrosis factor-α (TNF-α), chemokine (CC-motif) ligand 2 (CCL2), chemokine (CC-motif) ligand 3 (CCL3)), this issue remains largely unresolved and, once elucidated, may identify novel approaches to improve outcomes in COVID-19-infected patients (Nichols, JM., et al., Cannabis Cannabinoid Res., 5(1):12 (2020)).

The term “cannabinoid” as used herein may encompass a chemical compound that activates any mammalian cannabinoid receptor, for example human CB₁ receptor or human CB₂ receptor. The cannabinoids may be naturally occurring (such as, for example, endocannabinoids or phytocannabinoids) or they may be synthetic. Synthetic cannabinoids may include, for example, the classical cannabinoids structurally related to THC, the non-classical cannabinoids (cannabimimetics) including the aminoalkyindoles, 1,5-diarylpyrazoles, quinolines and arylsulphonoamides, and eicosanoids related to the endocannabinoids. When a cannabinoid salt is used, it may be employed in the form of a solution. The one or more cannabinoids is preferably selected from the classical cannabinoids, more preferably selected from tetrahydrocannabinols (THC), preferably delta-9-tetrahydrocannabinol and delta-8-tetrahydrocannabinol, cannabidiol (CBD), cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV and cannabigerol monomethyl ether (CBGM). CBD is a preferred cannabinoid.

Other cannabinoids suitable for use in the present invention are endocannabinoids, substances that naturally occur in the mammalian body and which activate one or more cannabinoid receptor. Preferably endocannabinoids are selected from arachidonoylethanolamine (AEA), 2-arachidonoylglycerol (2-AG), 2-arachidonyl glyceryl ether (noladin ether), N-arachidonoyl dopamine (NADA), virodhamine (OAE) and lysophosphatidylinositol (LPI).

Synthetic cannabinoids suitable for use in the present invention include nabilone, rimonabant, JWH-073, CP-55940, dimethylheptylpyran, HU-210, HU-331, SR144528, WIN 55,212-2, JWH-133, levonantradol, and AM-2201.

In recent years, cannabinoids have been investigated extensively due to their potential effects on the human body. Among all cannabinoids, Cannabidiol (CBD) has demonstrated a potent anti-inflammatory effect in a variety of inflammatory conditions. In one embodiment, CBD is used to contain the cytokine storm and treat the cytokine release syndrome associated with COVID-19 and other inflammatory viral conditions.

In another embodiment, the administration of CBD downregulates the level of pro-inflammatory cytokines and ameliorates the clinical symptoms of COVID-19. One embodiment provides a therapeutic role for CBD in the treatment of COVID-19 by reducing the cytokine storm, containing the damage, and re-establishing homeostasis.

### B. Intranasal Compositions

In one embodiment, the cannabidiol compositions are formulated to allow intranasal administration. Intranasal compositions may comprise an inhalable dry powder pharmaceutical formulation comprising a therapeutic agent, wherein the therapeutic agent is present as a freebase or as a mixture of a salt and a freebase. Pharmaceutical formulations disclosed herein can be formulated as suitable for airway administration, for example, nasal, intranasal, sinusoidal, peroral, and/or pulmonary administration. Typically, formulations are produced such that they have an appropriate particle size for the route, or target, of airway administration. As such, the formulations disclosed herein can be produced so as to be of defined particle size distribution.

For example, the particle size distribution for a salt form of a therapeutic agent for intranasal administration can be between about 5 µm and about 350 µm. More particularly, the salt form of the therapeutic agent can have a particle size distribution for intranasal administration between about 5 µ to about 250 µm, about 10 µm to about 200 µm, about 15 µm to about 150 µm, about 20 µm to about 100 µm, about 38 µm to about 100 µm, about 53 µm to about 100, about 53 µm to about 150 µm, or about 20 µm to about 53 µm. The salt form of the therapeutic agent in the pharmaceutical compositions of the invention can a particle size distribution range for intranasal administration that is less than about 200 µm. In other embodiments, the salt form of the therapeutic agent in the pharmaceutical compositions has a particle size distribution that is less than about 150 µm, less than about 100 µm, less than about 53 µm, less than about 38 µm, less than about 20 µm, less than about 10 µm, or less than about 5 µm. The salt form of the therapeutic agent in the pharmaceutical compositions of the invention can have a particle size distribution range for intranasal administration that is greater than about 5 µm, greater than about 10 µm, greater than about 15 µm, greater than about 20 µm, greater than about 38 µm, less than about 53 µm, less than about 70 µm, greater than about 100 µm, or greater than about 150 µm.

Additionally, the salt form of the therapeutic agent in the pharmaceutical compositions of the invention can have a particle size distribution range for pulmonary administration between about 1 µm and about 10 µm. In other embodiments for pulmonary administration, particle size distribution range is between about 1 µm and about 5 µm, or about 2 µm and about 5 µm. In other embodiments, the salt form of the therapeutic agent has a mean particle size of at least 1 µm, at least 2 µm, at least 3 µm, at least 4 µm, at least 5 µm, at least 10 µm, at least 20 µm, at least 25 µm, at least 30 µm, at least 40 µm, at least 50 µm, at least 60 µm, at least 70 µm, at least 80 µm, at least 90 µm, or at least 100 µm.

In some embodiments the disclosed cannabinoid compositions include one or more cannabinoids or pharmaceutically acceptable derivatives or salts thereof, a propellant, an alcohol, and a glycol and/or glycol ether. The alcohol may be a monohydric alcohol or a polyhydric alcohol, and is preferably a monohydric alcohol. Monohydric alcohol has a lower viscosity than a glycol or glycol ether. Accordingly, the composition is able to form droplets of a smaller diameter in comparison to compositions in which the monohydric alcohol is not present. The present inventors have surprisingly found that a specific ratio of monohydric alcohol to glycol or glycol ether results in a composition with a desired combination of both long term stability (for example the composition remains as a single phase for at least a week at a temperature of 2-40° C.) and small droplet size.

### C. Pulmonary Compositions

One embodiment provides a formulation and method for treating ARDS in the pulmonary system by inhalation or pulmonary administration. The diffusion characteristics of the particular drug formulation through the pulmonary tissues are chosen to obtain an efficacious concentration and an efficacious residence time in the tissue to be treated. Doses may be escalated or reduced or given more or less frequently to achieve selected blood levels. Additionally, the timing of administration and amount of the formulation is preferably controlled to optimize the therapeutic effects of the administered formulation on the tissue to be treated and/or titrate to a specific blood level.

Diffusion through the pulmonary tissues can additionally be modified by various excipients that can be added to the formulation to slow or accelerate the absorption of drugs into the pulmonary tissues. For example, the drug may be combined with surfactants such as the phospholipids, dimyristoylphosphatidyl choline, and dimyristoylphosphatidyl glycerol. The drugs may also be used in conjunction with bronchodilators that can relax the bronchial airways and allow easier entry of the antineoplastic drug to the lung. Albuterol is an example of the latter with many others known in the art. Further, the drug may be complexed with biocompatible polymers, micelle forming structures or cyclodextrins.

Particle size for the aerosolized drug used in the present examples was measured at about 1.0-5.0 µm with a GSD less than about 2.0 for deposition within the central and peripheral compartments of the lung. As noted elsewhere herein particle sizes are selected depending on the site of desired deposition of the drug particles within the respiratory tract.

Aerosols useful in the invention include aqueous vehicles such as water or saline with or without ethanol and may contain preservatives or antimicrobial agents such as benzalkonium chloride, paraben, and the like, and/or stabilizing agents such as polyethyleneglycol.

Powders useful in the invention include formulations of the neat drug or formulations of the drug combined with excipients or carriers such as mannitol, lactose, or other sugars. The powders used herein are effectively suspended in a carrier gas for administration. Alternatively, the powder may be dispersed in a chamber containing a gas or gas mixture which is then inhaled by the patient.

### III. Apelin

Apelin, an endogenous, multi-functional ligand for the G protein-coupled receptor, APJ, also serves as a second catalytic substrate for ACE2 (Chen. LJ, et al., Int J Hypertens. 2015:5 (2015)). Apelin is generated from a 77-amino acid precursor and undergoes proteolytic cleavage to generate biological active fragments, including apelin-36, apelin-19 and apelin-13. An endogenous protective role was postulated for activation of the apelin/APJ axis (Apelinergic system) after lung injury, via proposed mechanisms including suppression of the immune activating transcription factor, NF-κB and inhibition of innate immune infiltration/activation via attenuated expression of CCL2, CCL3, CCL4, CCL7 and TNF-α (Huang, S., et al., Clin Chim Acta., 456:81 (2016)). Of interest, both apelin and APJ are widely expressed throughout the lung, heart, liver, gut, kidney and central nervous system (Kawamata, Y., et al., Biochim Biophys Acta., 1538(2-3):162 (2001)), spatially overlapping expression of the endocannabinoid system while interaction between the endocannabinoid system and apelin limits liver fibrosis (Melgar-Lesmes, P., et al., Cells, 8:1311 (2019)). In one embodiment the regulation of the apelinergic system by CBD limits excessive pulmonary inflammation after ARDS.

### IV. Administration

Inhalation is a convenient administration route for therapeutic agents that overcomes many of the drawbacks of oral administration, such as slow drug onset and first-pass metabolism plus it can be used with patients that suffer from pulmonary conditions.

### A. Intranasal Administration

In one embodiment, the CBD compositions are delivered through intranasal administration. As described herein, intranasal administration or nose administration comprise the described compositions being administered into the mammal nostril and reaching nasal meatus or nasal cavity. For example, the compositions can be administered with nasal spray, insufflation, nasal drop, aerosol, propellant, pressurized dispersion body, aqueous aerosol, propellant, nose suspension, instillation, nasal gel, nose is with ointment and nose ointment, by means of any new or old type equipment of administration.

Current studies strongly suggest that intranasal administration of high dose Poly(I:C) in a murine model maybe a reliable working and practical model to investigate and help better understanding of mechanisms responsible for COVID-19 symptoms. Induction of hypo-oxygenation, lymphopenia, cytokine storm (marked production of proinflammatory cytokines) as well as impairment and destruction of lung architecture are all examples of cardinal clinical symptoms of severe COVID-19 and ARDS. In addition, being a synthetic analog of double stranded RNA, not only can Poly(I:C) provide a very robust foundation to study the immunopathology and physiology of COVID-19, but when compared to many viral agents, it is considerably safer and cheaper alternative for research on SARS-Cov-2 and other virus-induced infections.

Present findings propose a potential immunotherapeutic role for CBD in the treatment of severe respiratory viral infections and ARDS. The current data support the notion that anti-inflammatory function of CBD may be a crucial modulator of cytokine storm and hyperinflammation. There are reports suggesting that the interaction between immune system and COVID-19 is a two-phased process. The first stage is the activation of immune system to decimate the viruses and contain the progression of infection. The second stage is characterized by a regulatory mechanism to curtail the cytokine storm and prevent the cytokine-induced sepsis. The vast distribution of endocannabinoid system in the body is well documented and mounting evidence support anti-inflammatory effects for cannabinoids. Therefore, altogether, it maybe plausible to propose CBD as a potential immunomodulator in the treatment of COVID-19 and ARDS. In conclusion, these new findings introduce a new angle with a translational perspective to investigate the potentials of cannabinoids in the treatment of viral respiratory diseases such as COVID-19. Further studies are required to foster and validate such a complex therapeutic strategy in the treatment of severe viral respiratory infections such as COVID-19.

One embodiment provides a method of treating COVID-19 symptoms in a subject in need thereof by administering to the subject an effective amount of a composition including cannabidiol.

Another embodiment provides a method of reducing Acute respiratory distress syndrome in a subject in need thereof by administering to the subject an amount of cannabidiol effective to reduce inflammation in the subject. The cannabidiol reduces the level of inflammatory cytokines. The cytokines can be circulating cytokines or lung cytokines. In one embodiment, cannabidiol treatment reduces inflammatory damage to the lungs. In another embodiment, cannabidiol treatment improves the functional capacity of the lungs. Acute respiratory distress syndrome can be caused by COVID-19.

### B. Pulmonary Administration

In another embodiment, the cannabinoids are delivered through pulmonary administration directly to the lungs where they are efficiently absorbed into the systemic circulation, resulting in a rapid onset of therapeutic action. The rapid onset of therapeutic action achievable through the compositions and methods of the invention offers an advantage over prior cannabinoid delivery methods such as sublingual or suppository delivery, which generally involve slower systemic absorption.

Pulmonary administration by inhalation may be accomplished by means of producing liquid or powdered aerosols, for example, by using any of various devices known in the art. PCT Publication No. WO 92/16192 dated Oct. 1, 1992; PCT Publication No. WO 91/08760 dated Jun. 27, 1991; NTIS Patent Application 7-504-047 filed Apr. 3, 1990 by Roosdorp and Crystal) including but not limited to nebulizers, metered dose inhalers, and powder inhalers. Various delivery devices are commercially available and can be employed, e.g. Ultravent nebulizer (Mallinckrodt, Inc, St. Louis, Mo.); Acorn II nebulizer (Marquest Medical Products, Englewood, Colo.); Ventolin metered dose inhalers (Glaxo Inc., Research Triangle Park, N.C.); Spinhaler powder inhaler (Fisons Corp., Bedford, Mass.) or Turbohaler (Astra). Such devices typically entail the use of formulations suitable for dispensing from such a device, in which a propellant material may be present. Ultrasonic nebulizers may also be used.

As will be understood by those skilled in the art of delivering pharmaceuticals by the pulmonary route, a major criterion for the selection of a particular device for producing an aerosol is the size of the resultant aerosol particles. Smaller particles are needed if the drug particles are mainly or only intended to be delivered to the peripheral lung, i.e. the alveoli (e.g. 0.1-3 µm), while larger drug particles are needed (e.g. 3-10 µm) if delivery is only or mainly to the central pulmonary system such as the upper bronchi. Impact of particle sizes on the site of deposition within the respiratory tract is generally known to those skilled in the art.

### EXAMPLES

### Example 1 Cannabidiol as a Treatment Modality for COVID-19

### Materials and Methods:

Animal model and application of Poly(I:C) and CBD: Wild-type (WT) C57BL/6 mice (male, 12 weeks old) were divided into 3 experimental groups of sham, control and treatment (n=5). All animals were housed in pathogen-free conditions at the animal facility of the Augusta University, and all experiments were performed in accordance with the rules and regulations of the Augusta University Institutional Animal Care and Use Committee (IACUC). All mice were anesthetized with isoflurane. Sham group received PBS while control and treatment groups were administered by Poly(I:C) (Sigma Aldrich, USA) (100 µg in 50 µl sterile PBS) intranasally (I/N) three once-daily doses. CBD (isolate THC free) was delivered intraperitoneally (5 mg/kg), first dose two hours after the second Poly(I:C) treatment and every other day interval, total of 3 doses. Sham and control groups received PBS only. All mice were sacrificed at 8 days after the first Poly(I:C) application. Blood and lung tissues were harvested and subjected to the further analysis.

Measurement of vital signs: Vital signs including temperature, Blood O₂ saturation were measured prior and post of any treatment. Central body temperature was measured rectally and blood oxygen saturation was determined using portable pulse oximetry through carotid arteries.

Histology and immunohistochemistry: Left lobes of lung tissue were fixed in 10% neutral buffered formalin. Samples were processed by routine methods, oriented so as to provide coronal sections and 5 micron mid-coronal sections cut and stained with hematoxylin & eosin, Trichrome for histology. As for inflammatory indices, immunohistochemistry was performed by incubating the samples with specific antibodies against murine IL-6 (Cat# 554402, BD BioSciences Pharmingen) and Neutrophils (Cat#G102, Leinco Technologies). Preparations were counterstained with hematoxylin (catalog no. 7221; Richard-Allan Scientific, Kalamazoo, MI, USA) and mounted in Faramount (catalog no. S3025, DAKO), analyzed and imaged by brightfield microscopy.

Analytical Flow Cytometry: Single cell suspension was prepared from lung tissues. Briefly, tissue samples were sieved through a 100 µM cell strainer (BD Biosciences, San Diego, CA), followed by centrifugation (1,000 rpm, 10 min) to prepare single-cell suspensions. All cells were then stained with fluorescent antibodies to quantify neutrophils, macrophages, lymphocytes and cytokine expression. Briefly, all cells were stained with anti-Gr1 (Neutrophils), Anti-F4/80 (Macrophages), and anti-CD3/CD4/CD8 (Lymphocytes, all from Biolegend USA). Then cells were fixed and permeabilized and stained intracellularly for cytokines including IL-6, TNFα, IL-2, and IFNγ (Proinflammatory cytokines). All samples were run through a 4-Laser LSR II flow cytometer. Cells were gated based on forward and side scatter properties and on marker combinations to select cells of interest. All acquired flow cytometry data were analyzed using the FlowJo V10.

Statistical analysis: Graphs and summary statistics were also used to assess the results. All statistical tests were 2-sided. Except for where noted, all p-values presented are unadjusted for multiple comparisons.

### Results:

In the current study, to test the potential of cannabinoids in the treatment of viral infection and ARDS effects, Polyriboinosinic:polyribocytidylic acid [poly(I:C)] was used in a murine model to simulate the physiologic viral disease state and clinical symptoms of ARDS. Poly(I:C) is a stable synthetic double-stranded (dsRNA) compound that can replicate major effects of viral infections by binding to the Toll-Like receptor 3 (TLR3) with high affinity (11). Intranasal application of Poly(I:C) induced a significant inflammatory response and affected the functional capacity of the lung tissue and airways as seen in COVID-19 and ARDS.

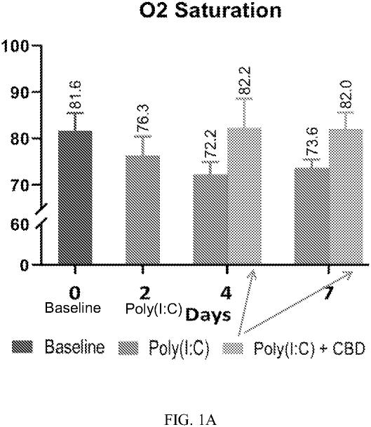

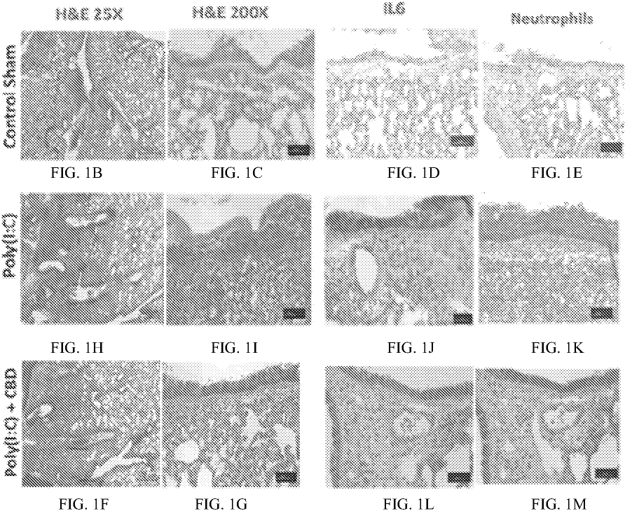

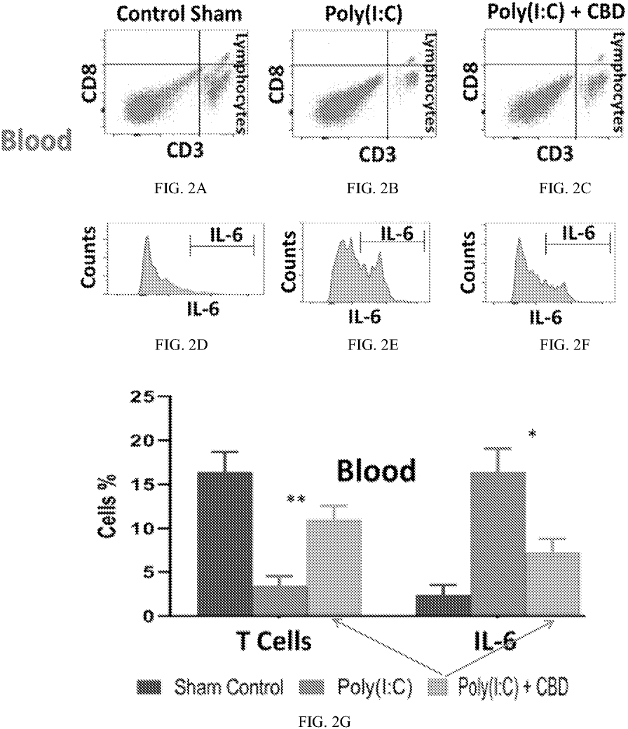

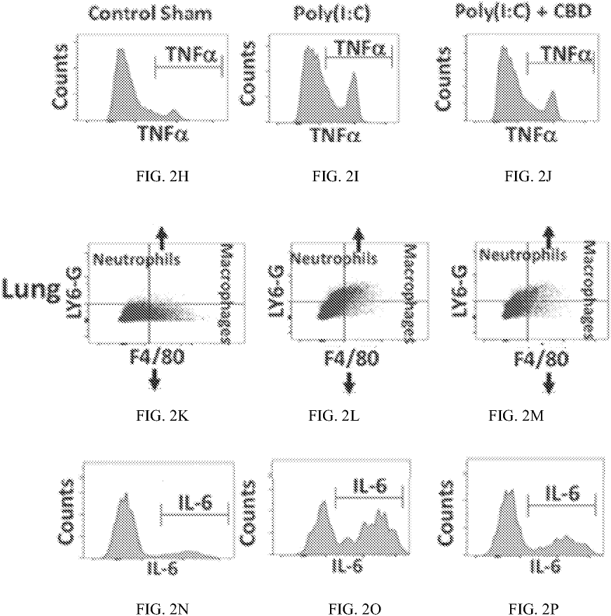

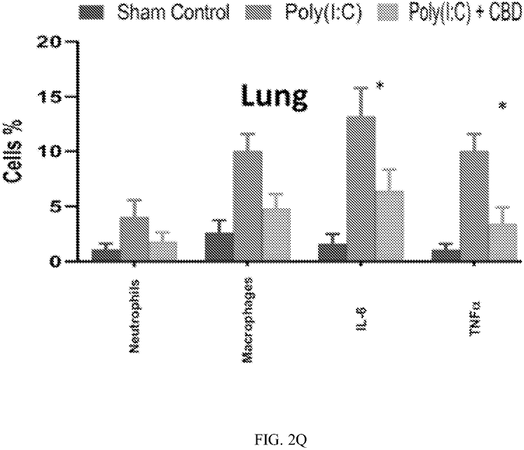

Poly(I:C) reduced the blood oxygen saturation by 10% (FIG. 1A) and the histological examination of lung tissues demonstrated that Poly(I:C) caused a significant perivascular and peri-bronchiolar interstitial inflammatory infiltrate compared to the normal tissue (FIGS. 1B-1E). Poly(I:C) produced structural damages to the lung including, but not limited to, fibrosis, hypertrophy and pulmonary edema evidenced by the widened interstitial space surrounding the airways and vasculature (FIGS. 1D-1E). These symptoms were totally or partially reversed and returned to the level and condition of the normal after treatment with CBD (FIGS. 1F-1G). Further, immunohistochemistry analysis of lung tissues revealed that Poly(I:C) treatment resulted in a marked increase in IL-6 and infiltrating neutrophils compared to the normal tissue (FIGS. 1H & 1J). CBD treatment reduced the expression of IL-6 and lowered the frequencies of neutrophils in the lung (FIG. 1L). Flow cytometry analysis of blood showed significant reduction in number of lymphocytes (severe lymphopenia) (p<0.01), mild reduction in neutrophils, marked increase in monocytes and significant increases in the level of IL-6 (p<0.03), IFNγ and TNFα after Poly(I:C) treatment compared to the normal tissues (FIGS. 2A-2G). Flow cytometry analysis of lung demonstrated an increase in the frequencies of infiltrating neutrophils (p<0.01), macrophages and significant elevation (p<0.01) in the expression of pro-inflammatory cytokines (e.g., IL-6, TNFα and IFNγ; FIGS. 2H-2Q). CBD treatment reversed all these inflammatory indices and partially re-established homeostasis. In the blood, CBD treatment enhanced the rate of lymphocyte frequencies markedly (p<0.01) while reducing the number of neutrophils and monocytes as well as the level of proinflammatory cytokines significantly (e.g., IL-6, IFNγ and TNFα). In the lung, CBD treatment downregulated the number of infiltrating neutrophils and macrophages markedly, and reduced the level of cytokines significantly (p<0.05).

### Example 2 Cannabidiol Modulation of Apelin in Acute Respiratory Distress Syndrome

### Materials and Methods:

Polyinosinic:polycytidylic acid (Poly(I:C)), a high molecular weight, synthetic analog of double stranded RNA (dsRNA), was used to recapitulate the histopathological, physiological and immune features of ARDS associated with SARS-CoV-2 infection (as described above), including low oxygen saturation, lymphopenia, elevated frequencies of neutrophils/monocytes, excess production of pro-inflammatory cytokines and destruction of lung morphology (Khodadadi, H., et al., J Cannabis Cannabinoid Res., 5(3):10 (2020); Stowell, NC., et al., Respir. Res., 1(10):43 (2009)). Adult (12 weeks) male C57Bl/6 mice were block randomized into one of three experimental groups (n = 10 mice/group) by a blinded investigator. Group I received intranasal, once daily administration of sterile saline for three consecutive days to serve as a control. Group II received intranasal, once daily administration of Poly I:C (100 µg in 50 µL in sterile saline) for three consecutive days to mimic ARDS. Group III received intranasal, once daily administration of Poly I:C (100 µg in 50 µL in sterile saline) for three consecutive days, with intraperitoneal administration of CBD (isolate CBD, THC-free, 5 mg/ kg body weight, Canabidiol Ltd, Dublin, Ireland), first dose two hours after the second Poly(I:C) treatment and every other day for a total of 3 doses to the treatment group. Blood oxygen saturation was quantified via the carotid arteries using a portable pulse oximeter at study initiation (day 0) and once daily for the duration of the study. Mice were euthanized at study day nine. Blood and lung tissue were harvested and subjected to flow cytometry, immunofluorescence and histological analysis, as detailed previously (Khodadadi, H., et al., J. Cannabis Cannabinoid Res., 5(3):10 (2020)). All flow cytometry data were analyzed using the FlowJo V10 software while immunofluorescence, and histological preparations were analyzed and imaged by fluorescence and bright field microscopy. As for additional histological evaluation, Masson’s Trichrome staining was used for the detection of collagen fibers in lung on formalin-fixed, paraffin-embedded sections. The collagen fibers stained in blue and the background is stained red. Sections were examined and analyzed using bright field microscopy imaging.

### Results:

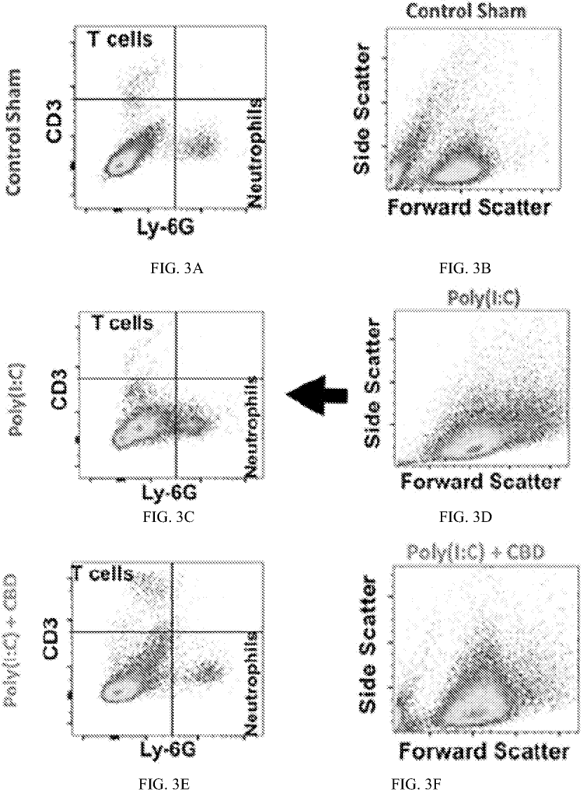

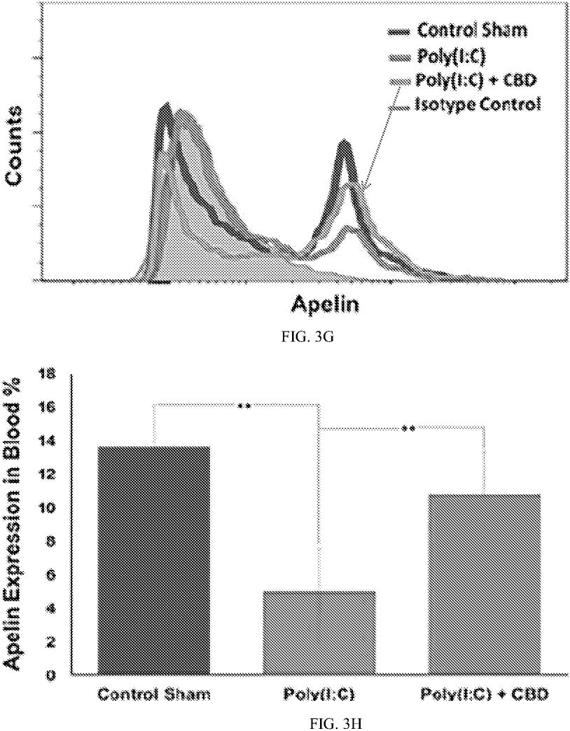

Flow cytometry analysis of whole blood showed that Poly(I:C)-treated mice (FIGS. 3C and 3D) exhibited a pattern of lymphopenia, lower frequency of T cells and elevated rate of neutrophils compared with the sham control group (FIGS. 3A and 3B). Further, Poly(I:C)-treated mice demonstrated significant reduction in the expression level of Apelin compared with the sham control group (FIG. 3G). Conversely, administration of CBD not only returned decreased T cells and increased neutrophils towards the normal level (FIGS. 3E and 3F), but also, enhanced expression of apelin in the blood following poly I:C treatment (FIG. 3H).

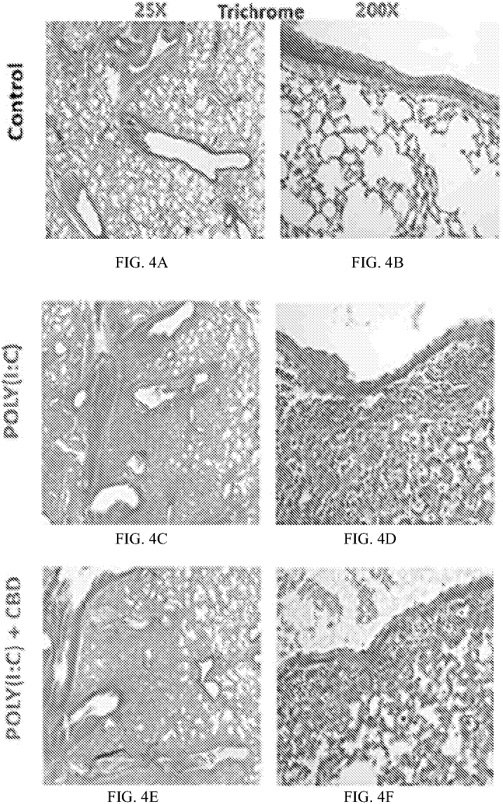

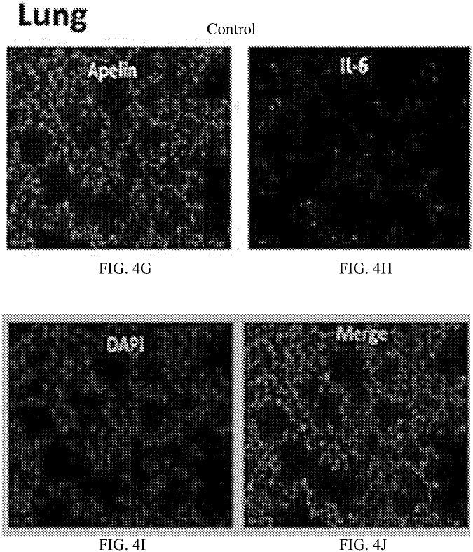

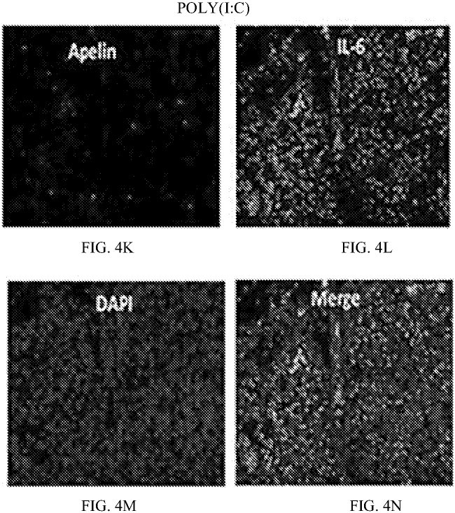

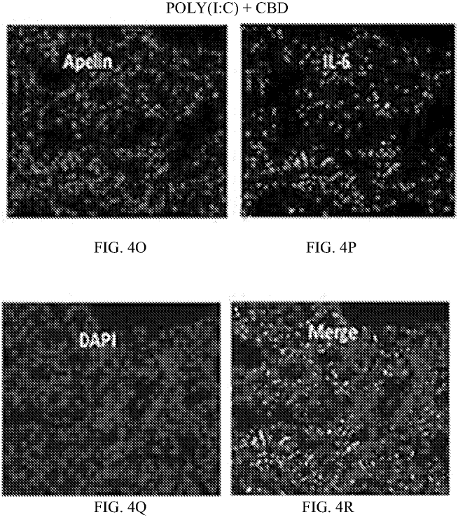

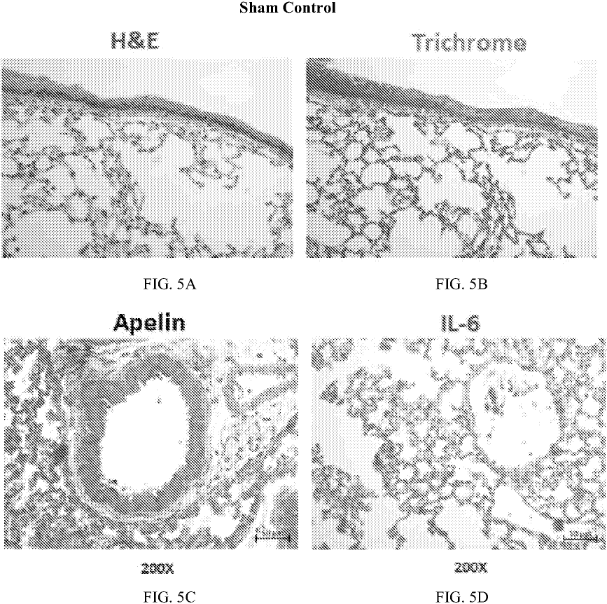

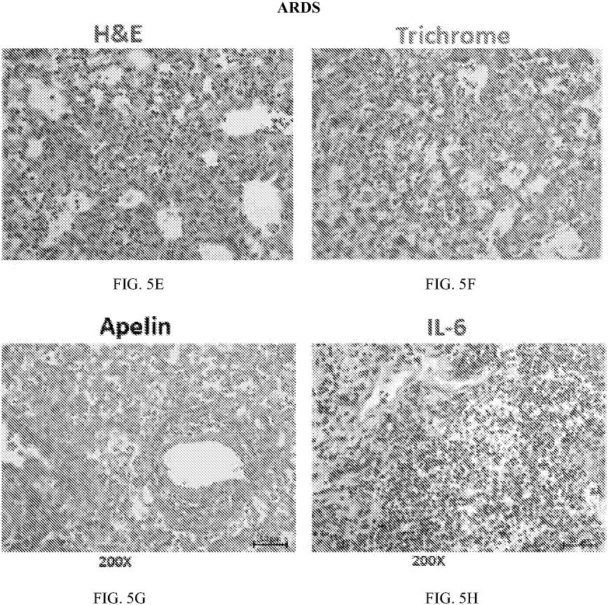

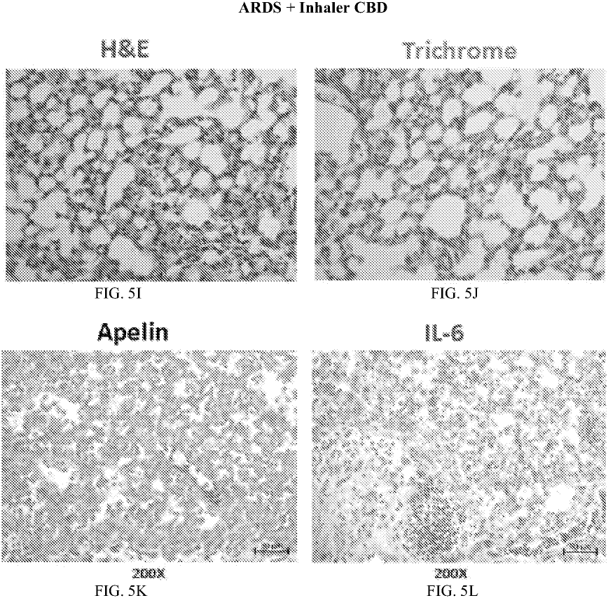

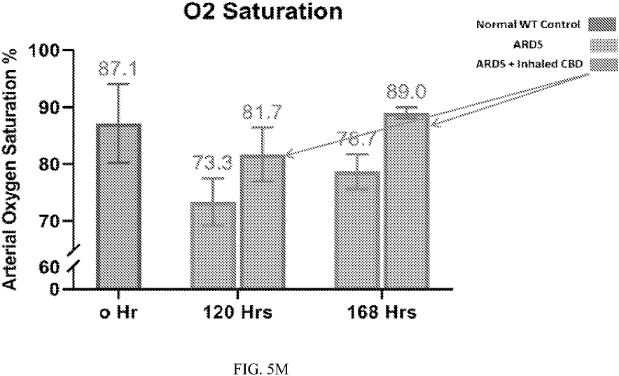

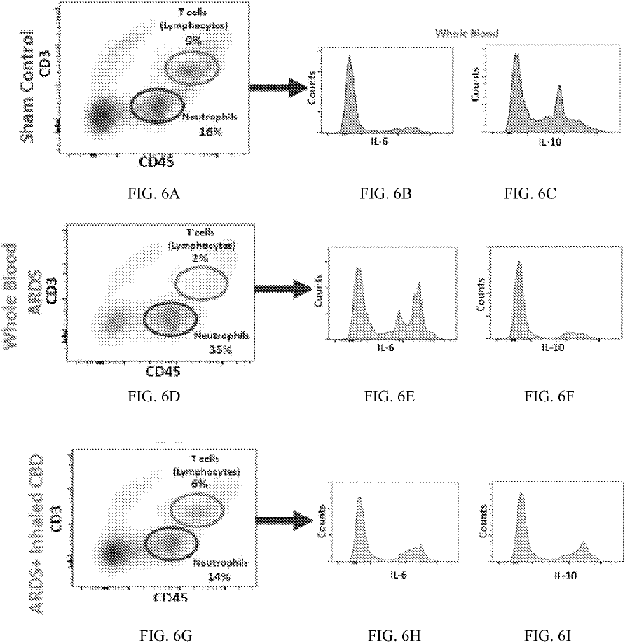

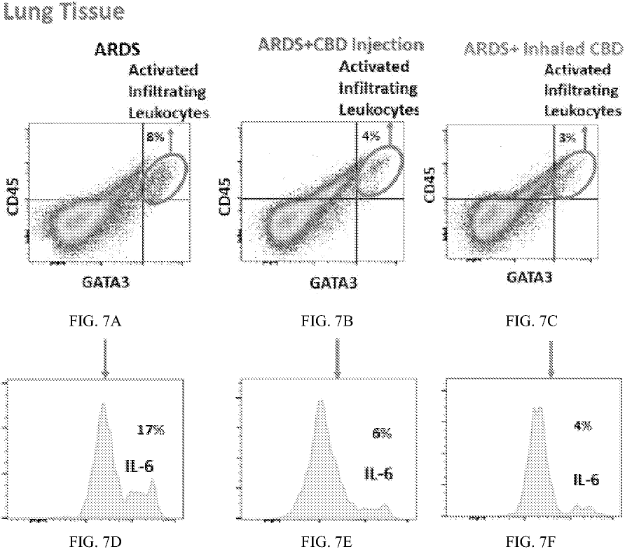

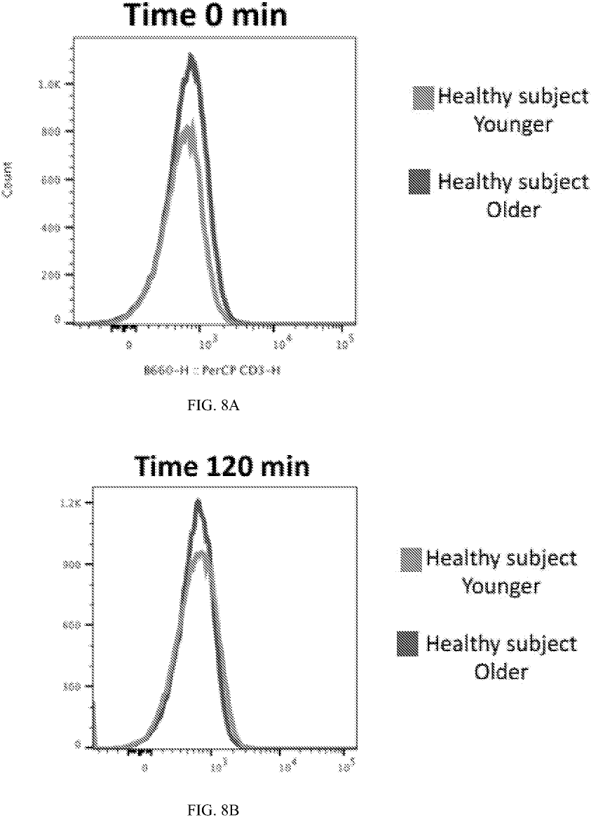

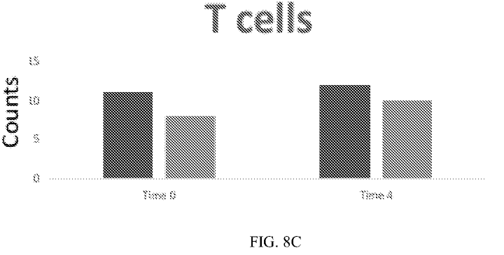

In addition, histological examination of lung tissues demonstrated that Poly(I:C) caused significant perivascular and peri-bronchiolar interstitial inflammatory infiltrate, fibrosis, hypertrophy and pulmonary edema, as evidenced by the widened interstitial space surrounding the airways and vasculature (FIGS. 4C and 4D). The pathological features of poly I:C administration were completely or partially abolished by following administration of CBD (FIGS. 4E and 4F). Immunofluorescence analysis of lung tissue revealed a reduction in apelin immunoreactivity after poly I:C treatment (FIG. 4K), as compared to control mice (FIG. 4G). Importantly, treatment with CBD increased apelin expression towards control levels in the lung following poly I:C administration (FIG. 4O).

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method of reducing inflammatory symptoms of COVID-19 in a subject in need thereof comprising administering to the subject an amount of cannabidiol effective to reduce inflammation in the subject.

2. The method of claim 1, wherein the subject has Acute respiratory distress syndrome wherein administration of the cannabidiol reduces pulmonary inflammation in the subject.

3. The method of claim 2, wherein the cannabidiol reduces the level of inflammatory cytokines in the subject compared to cytokine levels prior to administration of cannabidiol.

4. The method of claim 3, wherein the inflammatory cytokines are selected from the group consisting of IL-6, IFNγ and TNFα.

5. The method of claim 3, wherein the inflammatory cytokines are in the lung or airways.

6. The method of claim 2, wherein the cannabidiol treatment reduces inflammatory damage to the lungs.

7. The method of claim 2, wherein the cannabidiol treatment improves the functional capacity of the lungs.

8. The method of claim 2, wherein the acute respiratory distress syndrome is caused by coronavirus infection.

9. A pharmaceutical composition comprising an effective amount of a cannabinoid to reduce inflammation in a subject in need thereof.

10. The pharmaceutical composition of claim 8, wherein the composition is formulated for pulmonary, nasal, or aerosol administration.

11. (canceled)

12. (canceled)

13. The pharmaceutical composition of claim 8, wherein the cannabinoid is cannabidiol.

14. The pharmaceutical composition of claim 8, wherein the cannabinoid is selected from the group consisting of tetrahydrocannabinols (THC), delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV), cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

15. A method of reducing pulmonary inflammation in a subject in need thereof comprising administering to the subject an effective amount of a cannabinoid to reduce the pulmonary inflammation.

16. The method of claim 15, wherein the cannabinoid is cannabidiol.

17. The method of claim 15, wherein the cannabinoid is selected from the group consisting of tetrahydrocannabinols (THC), preferably delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV) and cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

18. The method of claim 15, wherein the subject is infected with a coronavirus.

19. The method of claim 18, wherein the coronavirus is SARS-CoV-2.

20. The method of claim 15, wherein the subject has ARDS.

21. The method of claim 15, wherein administering an effective amount of a cannabinoid futher reduces or treats a cytokine storm in a subject in need thereof.

22. (canceled)

23. (canceled)

24. (canceled)

25. (canceled)
